(19) United States
(12) Patent Application Publication
(10) Pub. No.: US 2011/0250687 A1
(43) Pub. Date: Oct. 13, 2011

(54) Cell adhesion inhibitor (CAI) with combination growth factors mobilization of peripheral blood mononuclear cells for CAI derived dendritic cell (CdDC) preparation and dendritic cell vaccine preparations generated from CdDC

(76) Inventors: Alex Wah Hin Yeung, Newport Beach, CA (US); Kevin James Yeung, Newport Beach, CA (US)

(21) Appl. No.: 12/798,743

(22) Filed: Apr. 12, 2010

(51) Int. Cl. C12N 5/078

(52) U.S. Cl. 435/378

(57) ABSTRACT

Disclosed is a method to recover a dendritic cell rich mixture from peripheral blood mononuclear cells (PBMC) mobilized with one or more cell adhesion inhibitors (CAI) for the preparation of a dendritic cell vaccine. The CAI derived dendritic cell rich mixture (CdDC) from PBMC can either be used alone or better still, be induced into dendritic vaccine specific preparations with the addition or modification with different antigens and methodologies of immunological induction methods known to the art. These CAI derived dendritic vaccines can then be used, but not exclusively so, in the treatment of cancer and infectious diseases. In order to achieve the best immature and mature dendritic cell rich mixture, peripheral blood cells mobilization may be achieved by administering, simultaneously or in sequence, to an individual one or more of a combination of different chemical compounds, hormones, growth factors etc. prior to PBMC collection with one or more of cell adhesion inhibitors such as a CXCR4 antagonist.

### BACKGROUND OF THE INVENTION:

Numerous clinical trials have demonstrated the safety of dendritic cells vaccines, and literally thousands of patients have received some form or another of dendritic cell vaccines with no serious adverse events. Notable clinical responses, though not always easy to come by, have been observed in about one half of the patients (Rideway 2003) (Banchereau 2001). A recent study showed that vaccination using dendritic cells loaded with four melanoma peptides (gp100, melan-A/MART-1, tyrosine melanoma antigen (MAGE-3), KLH and flu matrix resulted in regression of metastatic melanoma after four bimonthly vaccinations (Banchereau 2001). Another study for prostatic cancer, though not be able to demonstrate the intended delay of disease progression, demonstrated significant increase in median survival (Small 2007).

Dendritic cells (DCs) are considered the most potent antigen presenting cells (APCs) and thus play a crucial role in the stimulation of primary and secondary CD4+ and CD8+ T cell responses. Immature dendritic cells are characterized by efficient phagocytic activity for antigen up-take and processing within the cells. During the maturation process, DCs become less efficient in antigen capturing but more specialized in presenting immunogenic peptides and in activating naive T cells. For a DC vaccine to be successful, a combination of immature and mature cells and their synergistic action can be useful. DCs maturation can be mediated by inflammatory cytokines, or by additional stimuli such as CD40L, LPS or virus infection. All these stimuli add up to a rapid and sustained up-regulation of MHC class I antigen-processing machinery as well as of co-stimulatory molecules (CD40, CD80, CD86) and DC maturation marker CD83. Combination of these activation molecules is necessary for T-cell activation and the generation of cytotoxic CD8+ cells for the treatment of cancer.

During a viral infection or a malignant transformation, dendritic cells acquire antigens from the affected sites. These cells then migrate to the draining lymph nodes and present peptides associated MHC class I molecules, to helper CD4+ cells and CD8+ T cells. The way by which DCs phagocytose these foreign antigens from the extracellular environment and effectively present those selected peptides associated to MHC class I molecules, to CD8+ T cells, is called cross-presentation and is likely the most important mechanism for the priming of CD8+ T cells responses against foreign antigens.

The priming and expansion of antigen-specific CD8+ T cell response is a complex process involving concerted interactions between lymphocytes and other professional antigen-presenting cells. This process plays an important role in linking innate and adaptive immunity. The priming of antigen-specific CD8+ T cells requires recognition through the T cell receptor of peptide-MHC class I complexes on the surface of appropriate APCs, as well as interaction with the regulatory and suppressor cell system. However, suitable peptides may also be derived from exogenous antigens intersecting this pathway after endocytosis by APCs, in the cross-presentation process. DCs must undergo a special activation process or confirmation step in order to cross-prime CD8+ T cells (Blankenstein 2002). Cross-presentation of antigens by unconfirmed DCs may stimulate an abortive response that culminates in cross-tolerance (Belz 2002). Under pathological conditions, DCs are confirmed by engagement of surface CD40 by activated CD4+ helper T cells or by viral derived macromolecules, which can trigger DC maturation and up-regulate the expression of surface co-stimulatory molecules. It has been reported that only mature DCs, such as those obtained from culturing immature GM-CSF/IL-4 DCs with tumor necrosis factor (TNFalpha) and prostaglandin E2, are efficient antigen presenting cells (APCs) for cross-priming of exogenous antigens to CD8+ T cells (Schuler 2003) (Kaiser 2003).

Other studies demonstrated that DCs generated from human monocytes after a single-step 3-days culture in the presence of IFN- and GM-CSF, exhibit phenotypic and functional properties typical of activated partially mature DCs (Santini 2000). It is interesting to know that IFN induced DCs are more efficient than immature DCs, in inducing a Th-1 type immune response and CD8+ T cells response against defined antigens in a variety of models (Lapenta 2003) (Santodonato 2003) (Gabriele 2004) (L. a. Santini 2006). In this scenario, IFN, a well known drug with established clinical usage in human studies, may be one of the compounds that can be used, in addition to TNF alpha and prostaglandin E2, for the final maturation and cross priming of CD8+ cells. It addition, it is generally be assumed that CD34+ derived and mature DC is better than those coming from committed monocytes. These CD34+ derived mature DC can efficiently induce cross-priming of CD8+ T cells against exogenous antigens (LeBon 2002) (Blankenstein 2002) and can easily be mobilized using the standard G-CSF protocol.

So, as a summary of the current state of the art preparation of a DC vaccine from PBMC or otherwise, the first step is to culminate immature DCs which are active in phagocytic activity from several days of culture in the presence of GM-CSF/IL-4.

The second culture step, with the addition of activation factors such as IFN, TNF alpha and prostaglandin E2, more mature DCs that are capable of cross priming are generated (LeBon 2002) (Ridge 1998).

The third step is to incorporate the designed antigen or antigen inducing methodology into this DC vaccine preparation with or without the further addition of an adjuvant.

In order to circumvent regulatory and suppressor cell activity, recent trials have also include the use of molecules that can bypass or eliminate such activity. Administering monoclonal antibodies against the CTLA 4 receptor or against the CD25 molecule are two of such examples.

In order to achieve the three steps as mentioned above, the common method for preparing dendritic cells (DCs) is to collect peripheral blood mononuclear cells (PBMCs) from a subject, and then differentiate the monocytes, which are only a minute proportion of the PBMCs, into mature DCs in steps as described. The differentiation of PBMCs into DCs takes about one week, requires a GMP facility, and skilled technicians. Accordingly, providing facilities and personnel for manufacturing DC vaccines at or near each clinical site where PBMCs are obtained from a patient would likely be cost prohibitive.

Peripheral blood mononuclear cells (PBMC) usually are mobilized by growth factors acting on the bone marrow. The use of G CSF and GM CSF as mobilization agents for autologous stem cell support after high dose chemotherapy dated back nearly twenty years during the time when such therapy was popular with lymphomas and chemo-sensitive solid tumors such as late stage breast cancer (Yeung 1994). It is well known that a large number of hematopoietic CD34+ progenitor cells including a fair number of monocytes, from which the DC vaccine is derived, can be collected using a blood cell separator after 4 days of G CSF stimulation.

The present invention is to describe how, with the use of one or more cell adhesion inhibitors (CAI), that DC vaccine preparation can be done more easily and effectively.

The CAI derived dendritic cell (CdDC) preparation from this invention will be shown to have a rich population of both immature and mature DC that will be sufficient in number and active enough to be used either alone or be used by the addition of quick and easy ex vivo manipulations known to the art, thereby creating a standard platform from which DC and DC vaccine specific preparations can be made available as immunotherapy in cancer and infectious diseases.

### SUMMARY OF THE INVENTION

Plerixafor, a macrocyclic compound antagonist of the alpha chemokine receptor CXCR4, was approved by the FDA in late 2008 for hematopoietic stem cell (HSC) mobilization. The SDF-CXCL12/CXCR4 retention axis disruption by this agent in the bone marrow can release a whole host of progenitor cells without the necessity of priming. The result in HSC collection using G CSF and Plerixafor has been dramatic. Little, if any, is known of the effect of CXCR4 antagonist such as Plerixafor on the mobilization of monocytes or DC precursors into the peripheral blood.

This invention will show that, by using, in the priming process, a combination of growth factors for hematopoietic progenitor cells and dendritic cell activating and maturing agents, together with one or more cell adhesion inhibitors, will release these now committed progenitors from their niche. In addition, this synergistic priming strategy will be able to generate a sufficient number of activated immature and mature DCs in the peripheral blood that can be collected for immediate use.

The priming is done by giving an individual, simultaneously or in sequence, with one or a combination of more than one agents from a long list of compounds of hematopoietic growth factors and DC maturing agents such as G-CSF, GM-CSF, IL4, IFN, SCF, TNF alpha and prostaglandin E2 etc.

This invention thereby produces a unique cell adhesion inhibitor mobilized PBMC product that contains enough active, immature and mature DCs (CdDC).

This CdDC can then be harvested by the usual PBMC collection technique and system well known to those familiar with the art. The end product from this standardized technique will yield pharmaceutical grade materials that are immediately ready for DC vaccines preparation.

This invention will also show that the DCs in this CdDC preparation are equal or superior in effectiveness of immunological stimulation and chemotraction than those that are now being done by an ex vivo process. Studies have shown that culturing mononuclear cells ex vivo may change chemoreceptor expressions and chemotactic proteins secretion by these DCs (Fanutuzzi 1999).

In vivo priming, such as the one described for the generation of CdDC in the case of an autologous tumor vaccine preparation, may also have an added advantage that some of these antigen presenting cells, during the process of maturation, will be exposed to a full array of tumor antigens that are specific to that individual, further increasing the chance of a successful immunotherapeutic response later on.

CdDC can then be used to generate CdDC specific vaccines from a variety of antigen incubation or induction techniques that are well known to the art or be cryogenically preserved for easy future retreatment. Adjuvants may also be added to enhance the immunological response.

One of the most important advantages of this invention is that no GMP or clean room facility is required all along in the vaccine generation process. This new idea will enable the use of dendritic vaccine as a totally outpatient process, inexpensive, and available at all points of care facilities.

As a conclusion, the present invention will show that CdDC, by itself or by the CdDC vaccines generated from CdDC, may be used for a variety of cancers or infectious diseases, the medical practice of which is well known to those familiar in the art.

### DETAIL DESCRIPTION OF THE INVENTIONS

In a first aspect, the invention describes a method of preparation of a unique population of cell adhesion inhibitor derived dendritic cells (CdDC) derived from the peripheral blood mononuclear cells (PBMC) by the priming of an individual with one or a combination or more than one cell adhesion inhibitors such as Plerixafor (a CXCR4 antagonist).

In another aspect, an individual will be primed, simultaneously or in sequence, with one or a combination of more than one agents such as, but not exclusively limited to, flt3 ligand, G-CSF, GM-CSF, IL4, IFN, SCF, TNF alpha, prostaglandin E2, ILL 116, CD4OL, other hormones and growth factors etc. prior to the administration of the cell adhesion inhibitor.

In another aspect, other cell adhesion inhibitors can also be used alone by itself and by combination with others (Rideway 2003). The list includes but not limited to, besides a CXCR4 antagonist, compounds such as known proteases, e.g. neutrophil elastase and cathepsin G, which may be able to cleave VCAM-1, MMP-9 related molecules such as IL8, compounds that inhibit any chemokine ligand and receptor combinations, e.g. GROβ/CXCR2.

In another aspect, CdDC preparations will be shown to compose of adequate numbers of DC that contain active immature and mature cells with phenotypic characterization done by flow-cytometry consisting of expression patterns in CD80, CD86, CD11c. CD14, CD1a and CCR7 etc.

In another aspect, this invention will have shown the establishment of a unique end product of CdDC preparation without ex vivo manipulation that was previously unknown in the DC immunotherapy field.

This preparation of CdDC alone, without further ex vivo manipulation, may be able to use for treating diseases, e.g., if it can be shown to contain enough active cellular components that can be effective by itself in the induction of a cytotoxic immunologic response in certain types of cancer.

This preparation of CdDC, however, may need further ex vivo manipulation. Such ex vivo manipulation may include all existing and future methods that are known to the art in the preparation of DC vaccine such as, but not exclusively limit to, culturing with different agents such as GM-CSF, IL4, TNF alpha, prostaglandin E2, IFN, SCF, ILL IL6 and CD4OL etc. in one or multiple steps to achieve the effect of a CdDC vaccine specific preparation.

In another aspect and most likely so, this preparation of CdDC vaccine may need only one other simple procedure of antigen loading, thus avoiding the tedious and expensive step of ex vivo cell culturing. This simple antigen induction or incorporation technology is well known to those familiar with the art. Techniques that are commonly used are, including but not exclusively limited to, specific peptide incorporation such as Mage-1, Mage-3, gp-100 and MUC-1, irradiated tumor cells, tumor lysates or apoptotic tumor cells, DC tumor hybrids generated by electrofusion or polyethylene glycol, gene insertion with tumor associated antigen (TAA), tumor derived mRNA etc.

In another aspect, CdDC vaccine preparations may need the addition of a vaccine adjuvant, the selection of which from a long list of potential candidates is well known to those familiar with the art.

In a further aspect, the final CdDC vaccine preparation may be used in combination of other existing or future regulatory and suppressor cell elimination strategy, including but not exclusively limited to, therapies such as anti CTLA 4 or CD25 antibodies.

In another embodiment of this invention, leukapheresis is used to isolate CdDC from the peripheral blood using known blood cell separators (PBMC technique) that are now available in the commercial market. Examples are machines made by Haemonetics V50 blood separator, the Baxter CS 3000, the Fresenius AS 104 and the Fresenius AS TEC 104 and the Excel. By varying the separation method, leukapheresis can be adapted to isolate different cellular components from the peripheral blood. In the case of CdDC, the methodology employs those that separate the mononuclear cell fraction.

In a further aspect of this invention, PBMC technique is able to generate CDDC in a closed system by an outpatient facility. The final CdDC product is cleansed and further concentrated by density gradient centrifugation in an automatic process and be available for immediate use or be cryogenically stored for later use. Cryogenic preservation methods are well known in the art, some of which will be described below.

In another embodiment, cells in the CdDC can further be enriched differentially by those based on surface antigens expressed by certain types of DCs, e.g. using FACS so that the fractions of the different kinds of DCs in the CdDC can be altered. Alternatively, cells can be sorted by mixing with magnetic beads coated with monoclonal antibodies against a cell surface antigen characteristically expressed by stem cells. In summary, any known methodology in the art can be employed to further change the composition of the different cell types within the CdDC repertoire.

In another embodiment, CdDC from this invention relates to methods of autologous vaccination, infusion or implantation, meaning the use of tissues or cells from a subject's own body rather than from a donor individual.

The present invention also relates to methods of allogeneic vaccination. Allogeneic vaccination, infusion or implantation is the use of tissues or cells from a genetically non-identically individual of the same species.

The number, times and frequency of CdDC cells and vaccine preparation delivered to an individual vary depending on the disease and such information is well known to the art.

In a further embodiment for intravenous infusion, the CdDC cells can be placed in acceptable carriers with formulations well known in the art (e.g. Remington's Pharmaceutical Sciences 16^{th} edition, Osol, A. Ed. 1980). The cells are preferably being formulated in a solution with a pH from 6.5 to 8.5. Excipients to bring the cell mixture solution to isotonicity can also be added, such as 4.5% mannitol, normal 0.9% saline or sodium phosphate. Other pharmaceutically acceptable agents can also be added to bring the solution to isotonicity, including, but not limited to dextrose, boric acid, sodium tartrate, propylene glycol or other inorganic or organic solutes.

In another embodiment, the CdDC cells can be prepared as vaccine formulations. Methods for formulating dendritic cell vaccines are known to those of skill in the art. In a preferred embodiment, the CdDC cells are washed and resuspended in heat-inactivated plasma (preferably autologous plasma) and 10% dextrose at a concentration of 2×10⁹ cells/mi. The cells can then be diluted 1:1 with a mixture of heat-inactivated plasma and 20% DMSO to give a final concentration of 5% dextrose, 10% DMSO in heat-inactivated plasma. The target final filled formulation is 1×10⁹ cells/ml. in a container suitable for cyropreservation. The CdDC cells can then be administered to a patient or frozen, preferably at −80 degree C. for the duration of the first two cycles of vaccine administration. The rest may be stored in cryogenic freezer (preferably in a dry liquid nitrogen freezer designed to prevent contamination), preferably at a temperature of −150 degree C. The frozen vaccine may also be shipped to a clinical site for patient administration. Upon thawing, the vaccine can be administered directly to the patient without further processing.

In another embodiment using CDDC for short term treatment options, the remaining cells in CdDC, after properly prepared in aseptic technique, can be stored in refrigerators or freezers commonly used at points of care facilities for a period of time of not more than 7 days.

Other suitable formulations for administration besides those mentioned above can include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, immune-stimulants, cytokines and adjuvants.

In one aspect, the use of CdDC and CdDC vaccine preparations is for the treatment of cancer, including but not exclusively limited to, cancers such as melanoma, prostate cancer, breast cancer, renal cell carcinoma, colon cancer, AML, and CML.

In another aspect, the use of CdDC and CdDC vaccine preparations is for infectious diseases, including but not limited to, diseases such as HIV.

### EXAMPLES

### Example I

Treatment of an Advanced Colorectal Cancer Patient with Autologous Tumor Lysate Pulsed CdDC Vaccine

Autologous Tumor Culture.

After surgical resection, this colorectal patient's tumor sample was processed for tissue culture by mincing them with scissors and passing them through metal meshes of decreasing pore size. The cell suspension was then plated onto tissue culture flasks and grown in DMEM/F10 (Irvine Scientific, Santa Ana, Calif.) plus 10% FCS (Irvine Scientific) and 1% penicillin/streptomycin (Invitrogen, Carlsbad, Calif.). Tumor culture was done to ensure that a source be maintained for future testing assessments as well as another way to maintain new supply source of tumor lysate.

Preparation of Tumor Lysate.

Tumor samples from surgery were processed in the laboratory to produce single-cell suspensions as follows: the surgical specimen was washed thrice in dissection medium (HBSS+30 Mg/ml catalase+6.6 μg/ml desferoxamine+25 μg/ml N-acetyl cysteine+94 μg/ml cystine−2HCI+1.25 μg/ml superoxide dismutase+110 μg/ml sodium pyruvate+2.4 μg/ml HEPES+0.36% glucose+800 μM MgCl₂+100 units/ml Fungi-Bact). Then, the specimen was minced with scissors and passed through metal meshes of decreasing pore size (0.38 and 0.14 mm), followed by a nylon mesh with a pore size of 0.21 mm. Cells were lysed by four freeze (on liquid nitrogen)/thaw cycles (room temperature). Lysis was monitored by light microscopy, and larger particles were removed by centrifugation (1900 rpm for 10 min at 4° C.). Supernatants were then passed through a 0.2 μm filter, the protein concentration was determined by Bio-Rad protein assay, and aliquots were frozen at 80° C. until use.

Priming of Patients with Growth Factors

Patient was given GM-CSF at a dosage of 10 μg/kg by subcutaneous route per day for 7 days. In the second phase, the patient was given interferon β 1a at a dosage of 44 μg by subcutaneous route on days 5 and day 7.

Cell Adhesion Inhibitor

Patient was given Plerixafor (CXCR4 antagonist) at 0.24 mg/kg by the subcutaneous route at 9 pm on day 7.

CdDC Preparation

On day 8, at or around 9 am, mononuclear cells were isolated by leukapheresis. A Fenwal CS 3000 blood cell separator was used to harvest the mononuclear cell layer. Leukapheresis yielded about 2×10¹⁰ CdDCs. The exact CdDC cell numbes in the product pack was counted and 3 aliquots of 1×10⁹ CdDCs each were transferred into three separate bags using aseptic technique and stored at a temperature of −80 degree C. The rest of the CdDCs was cryopreserved under standard protocol as described previously.

CdDC Vaccine Phenotypic Evaluation.

CdDC were resuspended in PBS containing 2% fetal bovine serum (v/v) and stained with anti-CD14 FITC, anti-HLADR phycoerythrin (PE), and biotinylated anti-CD1a, CD80, CD83, anti-CD86, anti-CD54, and anti-CD40 antibodies (BD PharMingen).

Pulsing of CdDCs with Autologous Tumor Lysate.

On the day before each of the three CdDC vaccinations (days—1,13, and 27), one aliquot each of CdDC containing 1×10⁹ cells were washed in RPMI 1640 with autologous patient serum supplemented with 50 μg/ml autologous tumor lysate. The CdDCs with the tumor lysate were incubated overnight for 18 h at 37° C. on a tissue rotator.

Treatment Schedule

Vaccinations were administered three times at day 0, 14 and 28. The patient received 1×10⁹ CdDC vaccine cells intradermally (i.d.) in the upper leg, 5-10 cm from an inguinal lymph node. The same schedule of 3 bi-weekly intradermal vaccinations was repeated twice at intervals of 6 months in the absence of recurrent disease.

Clinical Follow-up

Clinical response consisted of history, physical examination, serum CEA-level, PET/CT scanning at 6-month intervals.

Bibliography

Banchereau. “Melanoma dendritic vaccine.” Cancer Research 61 (2001): 6451-58.

Belz. “The CD8a+dendritic cells is responsible for inducing peripheral self intolerance to tissue associated antigens.” J. Exp. Med, 2002: 1099-1104.

Blankenstein. “Cross priming versus cross tolerance: are two signals enough?” Trends in Immunol, 2002: 171-173.

Fanutuzzi. “Loss of CCR2 expression and functional response to monocyte chemotactic protein MCP-during the differentiation of human monocytes.” Blood, 1999: 875-883.

Gabriele. “IFN alpha promotes differentiation of monocytes from patients with CML into activated dendritic cells mature after LPS.” Blood, 2004: 980-987.

Kaiser. “Naive CD8+ T cells recruitment and proliferation are dependent on stage of dendritic cells maturation.” Eur. J Immunol, 2003: 162-171.

Lapenta. “Potent immune response against HIV-1 and protection from virus challenge in hu-PBL-SCID mice immunized with inactivated virus pulsed dendritic cells generated in the presence of IFN-alpha.”J Exp Med, 2003: 361-367.

LeBon. “Links between innate and adaptive immunity via type 1 interferon.” Curr. Opin Immunology, 2002: 432-436.

Rideway. “dendritic cell vaccines (response rate).” Cancer Investigations 21 (2003): 873-876.

Ridge. “A conditioned dendritic cells can be a temporal bridge between a CD4+ T-helper and a T-killer cell.” Nature, 1998: 474-478.

Santini. “Type I interferon as a powerful adjuvant for monocyte derived dendritic cell development and activity in vitro and in HuPBL-SCID mice.” J. Exp. Med, 2000: 1777-1788.

Santini, Lapenta and. “IFN alpha conditioned dendritic cells efficient in cross priming CD8+ cells against viral antigens.” Eur J Immunol, 2006: 2046-2060.

Santodonato. “Monocyte derived dendritic cells generated after a short term culture with IFN and GMCSF stimulate a potent EBV specific CD8+ response.”J Immunol, 2003: 5195-5202.

Schuler. “Use of dendritic cells in cancer immunotherapy.” Curr. Opon Immunol, 2003: 138-147.

Small. “Provenge Survival.” Dendreon D9001 and 9002 trials, 2007.

Yeung, Alex W. “Double cycle high dose chemotherapy with peripheral blood stem cell and hematopoietic growth factor support in patients with advanced solid tumor.” Cancer 73, no. 7 (1994): 1960-1970.

1. A method of mobilization and then collection from the peripheral blood of a subject a population of peripheral blood mononuclear cells as a cell adhesion inhibitor derived dendritic cell (CdDC) preparation by firstly administering an effective amount, simultaneously or in sequence, one or more than one agents from a list of growth factors, hormones, chemicals or compounds including but not exclusively limited to flt3 ligand, G-CSF, GM-CSF, IL4, IFN, SCF, TNF alpha, prostaglandin E2, ILL IL6, CD40L etc. and secondly administering an effective amount of one or a combination or more than one from the category of cell adhesion inhibitor compounds, an example of which is a CXCR4 antagonist such as Plerixafor.

2. The method of claim 1, wherein the CdDC preparation and any ultimate CdDC vaccine preparations deriving from the CdDC preparation are for the treatment of cancer.

3. The method of claim 1, wherein the CdDC preparation and any ultimate CdDC vaccine preparations deriving from the CdDC preparation are for the treatment of infectious diseases.

4. The method of claim 1, wherein the CdDC vaccine preparation requires antigen induction or incorporation technology that is well known to those familiar with the art. Techniques that are commonly used are, including but not exclusively limited to, specific peptide incorporation such as Mage-1, Mage-3, gp-100 and MUC-1, irradiated tumor cells, tumor lysates or apoptotic tumor cells, DC tumor hybrids generated by electrofusion or polyethylene glycol, gene insertion with tumor associated antigen (TAA), tumor derived mRNA etc.

5. The method of claim 1, wherein the CdDC vaccine preparation may need the addition of a vaccine adjuvant, the selection of which from a long list of potential candidates is well known to those familiar with the art.

6. The method of claim 1, wherein the CdDC vaccine preparation may be used in combination with a regulatory or suppressor cell elimination strategy, includes but not exclusively limited to, therapies such as anti CTLA 4 or CD25 antibodies.

7. The method of claim 1, wherein the CdDC vaccine is used for an autologous treatment program.

8. The method of claim 1, wherein the CdDC vaccine is used for an allogeneic treatment program.
